(19) United States
(12) Patent Application Publication
(10) Pub. No.: US 2006/0068455 A1
(43) Pub. Date: Mar. 30, 2006

(54) GABA detection methods and applications

(76) Inventors: Seok Won Yim, Los Angeles, CA (US); Bruce S. Dunn, Los Angeles, CA (US)

Correspondence Address: SMITH, GAMBRELL & RUSSELL, LLP (UC);SUZANNAH K. SUNDBY, 1850 M. STREET NW, # 800, WASHINGTON, DC, 20036, US

(21) Appl. No.: 11/237,689

(22) Filed: Sep. 29, 2005

Related U.S. Application Data
(60) Provisional application No. 60/614,510, filed on Sep. 30, 2004.

(51) Int. Cl. C12Q 1/48; C12Q 1/32

(52) U.S. Cl. 435/15; 435/26

(57) ABSTRACT

Disclosed herein are silica glass sensors comprising more than one active biological material in a single sol-gel layer. The silica glass sensors may be used to assay complex biological mechanisms in cells and tissues cultivated thereon. Also disclosed are silica glass sensors having GABA-T, SSADH, or both, for detecting GAD, GABA, or SSA.

### CROSS REFERENCE TO RELATED APPLICATIONS

This application claims the benefit of U.S. Provisional Patent Application No. 60/614,510, filed 30 Sep. 2004, pending, and is herein incorporated by reference in its entirety.

### BACKGROUND OF THE INVENTION

1. Field of the Invention

The present invention generally relates to a sol-gel biosensor and methods of making and using thereof. In particular, the present invention provides a sol-gel biosensor for the real-time detection of γ-aminobutyric acid (GABA).

2. Description of the Related Art

γ-aminobutyric acid (GABA) is a major inhibitory neurotransmitter and is related with many diseases such as epilepsy, Huntington's disease, Parkinson's disease, diabetes, spinal cord injury, and so its accurate detection is of great interest. GABA has been detected mainly with chromatographic methods known in the art. GABA was separated and detected by liquid chromatography using derivatization with 2,4,6-trinitrobenzenesulfonic acid, anion exchange chromatography, or high performance liquid chromatography (HPLC) using o-phthaldialdehtde/2-mercaptoethanol (OPT) derivatization. See e.g. Caudill, et al. (1982) J. Chromatogr. 227:331-339; Wu, et al. (1979) Neurochem. Res. 4:201-212; and Lindroth, et al. (1979) Anal. Chem. 51:1667-1674. GABA localized in brain has been measured with ¹H Nuclear magnetic resonance (NMR) spectroscopy. See Rothman, et al. (1993) PNAS USA 90:5662-5666. Unfortunately, the direct and real-time detection of GABA is difficult because GABA is insensitive to electrochemical and spectrophotometrical methods. U.S. Patent Application Publication No. 20050173267 discloses a cumbersome gadget, an acoustical immunosensor, having a plurality of electrodes, piezoelectric materials, impedance analyzers, and the like for the real-time detection of GABA.

Thus, a need still exists for accurate and practical assay methods for GABA.

### SUMMARY OF THE INVENTION

The present invention generally relates to methods and devices for assaying complex cellular pathways including the γ-aminobutyric acid (GABA).

In some embodiments, the present invention provides a porous, transparent sol-gel glass having two or more active biological agents entrapped therein. In some embodiments, the active biological agents are enzymes. In some embodiments, the enzymes are involved in a given cellular or biological pathway. In some embodiments, the active biological agents are GABA-transaminase (GABA-T) and SSA-dehydrogenase (SSADH).

In some embodiments, the present invention provides a method for assaying a test sample for a substance which reacts with or whose reaction is catalyzed by an active biological material which comprises contacting a porous, transparent sol-gel glass having the biological material entrapped therein into contact with the test sample; and observing any change in the optical characteristics of the porous, transparent sol-gel glass. In some embodiments, the change in optical characteristics is observed using spectroscopic techniques selected from the group consisting of ultraviolet, infrared, visible light, fluorescence, luminescence, absorption, emission and reflection techniques. In some embodiments, the active biological material is GABA-transaminase (GABA-T), SSA-dehydrogenase (SSADH), or both. In some embodiments, the test sample is a cell or a tissue. In some embodiments, the cell or tissue is cultivated on the porous, transparent sol-gel glass having the biological material entrapped therein. In some embodiments, the substance is γ-aminobutyric acid (GABA), glutamic acid dehydrogenase (GAD), succinic semialdehyde (SSA), or a combination thereof.

In some embodiments, the present invention provides an assay for a substrate involved in the γ-aminobutyric acid (GABA) pathway in a sample which comprises contacting the sample with an enzyme for the substrate and observing conversion of the substrate. In some embodiments, the substrate is γ-aminobutyric acid (GABA), or succinic semialdehyde (SSA). In some embodiments, the enzyme is GABA-transaminase (GABA-T) or SSA-dehydrogenase (SSADH). In some embodiments, conversion of the substrate is observed by assaying the amount NADPH produced after the enzyme is contacted with the substrate. In some embodiments, the substrate is γ-aminobutyric acid (GABA) and conversion is observed by assaying the amount of succinic semialdehyde (SSA) produced after the enzyme is contacted with the substrate.

In some embodiments, the amount of succinic semialdehyde (SSA) is assayed by reacting with 4-amino-3-hydrazino-5-mercapto-1, 2, 3-triazole.

It is to be understood that both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1A schematically shows GABA-T catalyzes succinic semialdehyde (SSA) production from GABA and SSA then reacts stoichiometrically with 4-amino-3-hydrazino-5-mercapto-1, 2, 3-triazole (Purpald).

FIG. 1B is a calibration curve which shows that spectrophotometric detection with Purpald using the GABA-T sensor only accounts for some of the actual GABA present as evidenced by the GABA-T/SSADH sensor.

FIG. 2 is a calibration curve showing the amount of fluorescence for GABA using the GABA-T/SSADH silica glass sensor.

FIG. 3A shows EGFP fluorescence from transfected CHO cells on a plastic culture vessel.

FIG. 3B shows EGFP fluorescence from vector rGAD₆₇-EGFP transfected CHO cells on the silica glass sensor.

FIG. 3C also shows EGFP fluorescence from vector rGAD₆₇-EGFP transfected CHO cells on the silica glass sensor.

FIG. 4 shows that the GABA-T/SSADH silica glass sensor detected the fluorescence from NADPH in real-time, which is generated from degradation reaction of GABA produced by GAD₆₇ in CHO cells.

FIG. 5A evidences that the fluorescence from the GABA-T/SSADH sensors were the result of degradation reaction of GABA produced by GAD₆₇ in transfected CHO cells.

FIG. 5B confirms that the fluorescence from the GABA-T/SSADH sensor is due to degradation reaction of GABA produced by GAD₆₇ in CHO cells.

FIG. 6 shows the GABA-T/SSADH fluorometric silica glass sensor (rat neural tissue sections).

FIG. 7 is a graph of the fluorescence from degradation reaction of GABA released from the tissue sections.

FIG. 8 shows real-time fluorescence detection of rat brain slice which indicates that at 100 mM Ca²⁺, rat brain tissue released more GABA than controls.

FIG. 9 shows that 75 μM 2-APB without 10 mM CaCl₂ had about 41.9% less GABA release than 75 μM 2-APB with 10 mM CaCl₂, thereby indicating that 2-APB inhibits Ca²⁺-dependent GABA release and 10 mM CaCl₂ can modulate the inhibition of 2-APB.

FIG. 10 shows the effect of Thapsigargin on Ca²⁺-dependent GABA release.

FIG. 11 shows the effect of Ryanodine on Ca²⁺-dependent GABA release.

FIG. 12 shows an example of a silica glass sensor design for assaying the effect an agent has on cell growth and differentiation.

FIG. 13 shows an alternative silica glass sensor design for conducting comparative studies different agents have on cell growth and differentiation.

### DETAILED DESCRIPTION OF THE INVENTION

The present invention provides spectrophotometric and fluorometric silica glass sensors for assaying γ-aminobutyric acid (GABA), glutamic acid dehydrogenase (GAD), and succinic semialdehyde (SSA) and methods of making and using thereof.

As used herein, “assaying” is used interchangeably with “detecting”, “measuring”, “monitoring” and “analyzing”.

GAD catalyses α-decarboxylation from L-glutamic acid to produce GABA. GABA-transaminase (GABA-T) converts GABA to SSA. SSA-dehydrogenase (SSADH) converts SSA to succinate and the by-product, NADPH. As provided herein, SSA may be spectrophotometrically detected by reaction with 4-amino-3-hydrazino-5-mercapto-1, 2, 3-triazole (Purplad) and NADPH may be fluorometrically measured by its fluorescence.

1. Silica Glass Sensors

The enzymes used for the silica glass sensors exemplified herein were recombinant Escherichia coli GABA-T and SSADH. However, it is noted that other enzymes commercially available or obtained by methods known in the art may be used.

E. coli gabT and gabD genes were isolated and amplified by Polymerase Chain Reaction (PCR) from genomic DNA of E. coli strain DH5α using methods known in the art. See e.g. Hanahan, D. (1965) DNA Cloning: A practical approach, Vol. 1. E. coli DH5α genomic DNA was prepared using AquaPure Genomic DNA Isolation Kits (Bio-Rad, Hercules, Calif.).

The oligonucleotides for gabT (GABA-T) PCR
reaction were:

| | |
|---|---|
| 5′-CCCGGATCCATGAACAGCAATAAAGAGTT-3′ | (SEQ ID NO:1) |
| 5′-CCCAAGCTTCTACTGCTTCGCCTCATC-3′ | (SEQ ID NO:2) |

The oligonucleotides for gabD (SSADH) PCR
reaction were:

| | |
|---|---|
| 5′-CCCGGATCCATGAAACTTAACGACAGTAA-3′ | (SEQ ID NO:3) |
| 5′-CCCAAGCTTTTAAAGACCGATGCACATAT-3′ | (SEQ ID NO:4) |

The PCR reaction with E. coli DH5α genomic DNA was performed as pre-cycle step of 94° C. 2 minutes, 35 cycles of 94° C. 45 seconds, 50° C. 1 minute and 68° C. 2 minutes, post-cycle step of 68° C. 10 minutes. The PCR amplified gabT and gabD were cloned with pRSET A vector (Invitrogen, Carlsbad, Calif.) using methods known in the art. Recombinant E. coli GABA-T and SSADH were overexpressed separately in E. coli strain BL21(DE3)pLysS (Invitrogen, Carlsbad, Calif.) at about 15 to about 25° C. for about 10 to about 20 hours with 1 mM isopropylthiogalactopyranoside (IPTG). Recombinant E. coli GABA-T and SSADH were purified with Ni-NTA resin (Qiagen, Hilden, Germany) after E. coli was disrupted by sonication on ice.

For cloning GABA-T, the nucleic acid sequence having accession number M88334 (GI:147900) was used, however, it is noted that other suitable sequences and methods known in the art may be used. See e.g. Niegermann, et al. (1993) Arch. Microbiol. 160:454-460, which is herein incorporated by reference. The accession numbers for the amino acid sequences of the recombinant GABA-T and recombinant SSADH used herein are GI:147902 and GI:147901, respectively.

Sensor A. GABA-T—Spectrophotometric Silica Glass Sensor

A silica glass sensor comprising GABA-T was prepared according to the sol-gel method as disclosed in U.S. Pat. No. 5,200,334 and Ellerby, et al. (1992) Science 255:1113-1115, which are herein incorporated by reference. The sol was made by mixing 80.7% v/v tetramethylorthosilicate (TMOS >99%) (Aldrich, Milwaukee, Wis.) 18.1% v/v deionized water, and 1.2% v/v HCl (0.04 M) and sonicating for 15 minutes. The sol was mixed with 0.1M potassium phosphate buffer, pH 6.0 (87.7% 0.2 M KH₂PO₄ and 12.3% 0.2 M K₂HPO₄) and 30 ng to about 300 ng GABA-T per μl buffered sol. The buffered silica sol GABA-T mixture was poured into a cast. After 30 minutes gelation, the silica glass sensor was washed with phosphate-buffered saline (PBS, 137 mM NaCl, 2.7 mM KCl, 4.3 mM Na₂HPO₄.7H₂₀, 1.4 mM KH₂PO₄) and aged at 4° C. for more than about 12 hours. Then the silica glass sensor was washed several times with PBS before use. The amount of TMOS was about 24 μl in total 100 μl of buffered sol. The amount of enzyme was about 100 ng/μl buffered sol.

Sensor B. GABA-T/SSADH—Fluorometric Silica Glass Sensor

A silica glass sensor comprising GABA-T and SSADH was prepared according to the sol-gel method as disclosed in U.S. Pat. No. 5,200,334 and Ellerby, et al. (1992) Science 255:1113-1115, which are herein incorporated by reference. The sol was made by mixing 80.7% v/v tetramethylorthosilicate (TMOS >99%) (Aldrich, Milwaukee, Wis.) 18.1% v/v deionized wather, and 1.2% v/v HCl (0.04 M) and sonicating for 15 minutes. The sol was mixed with 0.1M potassium phosphate buffer, pH 6.0 (87.7% 0.2 M KH₂PO₄ and 12.3% 0.2 M K₂HPO₄) and 30 ng to about 300 ng GABA-T and SSADH per μl buffered sol. The buffered silica sol GABA-T/SSADH mixture was poured into a cast. After 30 minutes gelation, the silica glass sensor was washed with phosphate-buffered saline (PBS, 137 mM NaCl, 2.7 mM KCl, 4.3 mM Na₂HPO₄.7H₂O, 1.4 mM KH₂PO₄) and aged at 4° C. for more than about 12 hours. Then the silica glass sensor was washed several times with PBS before use. The amount of TMOS was about 24 μl in total 100 μl of buffered sol. The amount of enzyme was about 100 ng/μl buffered sol.

As used herein, “GABA-T sensor”, “GABA-T silica glass sensor” and “spectrophotometric silica glass sensor” are used interchangeably to refer to a silica glass sensor having only encapsulated GABA-T according to the procedure for Sensor A above.

As used herein, “GABA-T/SSADH sensor”, “GABA-T/SSADH silica glass sensor” and “fluorometric silica glass sensor” are used interchangeable to refer to a silica glass sensor having both GABA-T and SSADH encapsulated therein according to the procedure for Sensor B above.

As used herein, “silica glass” refers to a silica glass which does not contain any GABA-T or SSADH encapsulated therein. Such silica glasses are used as controls in some of the experiments described herein.

2. Detection

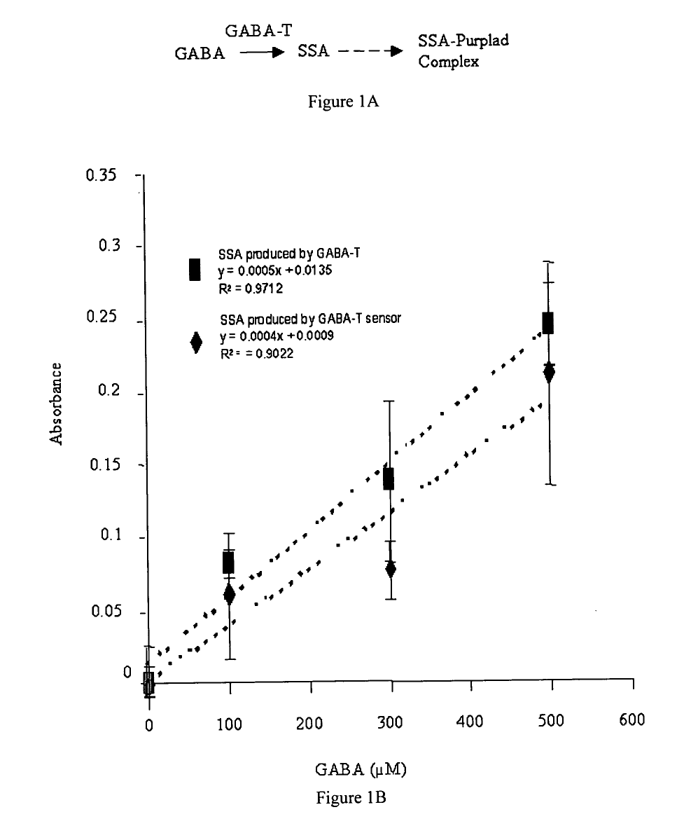

Purpald (4-amino-3-hydrazino-5-mercapto-1, 2, 4-triazole) reacts specifically with aldehydes to produce purple products. GABA-T catalyzes succinic semialdehyde (SSA) production from GABA and SSA then reacts stoichiometrically with Purpald as schematically shown in FIG. 1A.

A. Spectrophotometric Detection

After gelation and aging, the GABA-T silica glass sensor was incubated with GABA for 1 hour at 37° C. 1 mM α-ketoglutarate and 1.4 mM reduced β-nicotinamide adenine dinucleotide phosphate (β-NADP⁺, Sigma, St. Louis, Mo.) were added as co-substrates. GABA was determined with 80 mM Purpald solution and 0.23 N NaOH at room temperature. The absorbance of the purple color was measured at 513 nm with SPECTRA mad 340PC (Molecular Devices, Calif.). The Purpald reaction was preformed for 30 minutes with PBS.

B. Fluorometric Detection

After gelation and aging, the GABA-T/SSADH silica glass sensor was incubated with GABA for 1 hour at 25° C. or 37° C. 1 mM α-ketoglutarate and 1.4 mM reduced β-nicotinamide adenine dinucleotide phosphate (β-NADP+, Sigma, St. Louis, Mo.) were added as co-substrates. Fluorescence of NADPH was measured with Fluoroskan Ascent (Labsystems, Helsinki, Finland). Excitation and emission wavelength of NADPH was 355 nm and 460 nm at about 25° C. or 37° C.

FIG. 1B is a calibration curve which shows that spectrophotometric detection with Purpald using the GABA-T sensor only accounts for some of the actual GABA present as evidenced by the GABA-T/SSADH sensor. FIG. 1B also indicates that GABA and SSADH retain their activities in both GABA-T and GABA-T/SSADH silica glass sensors.

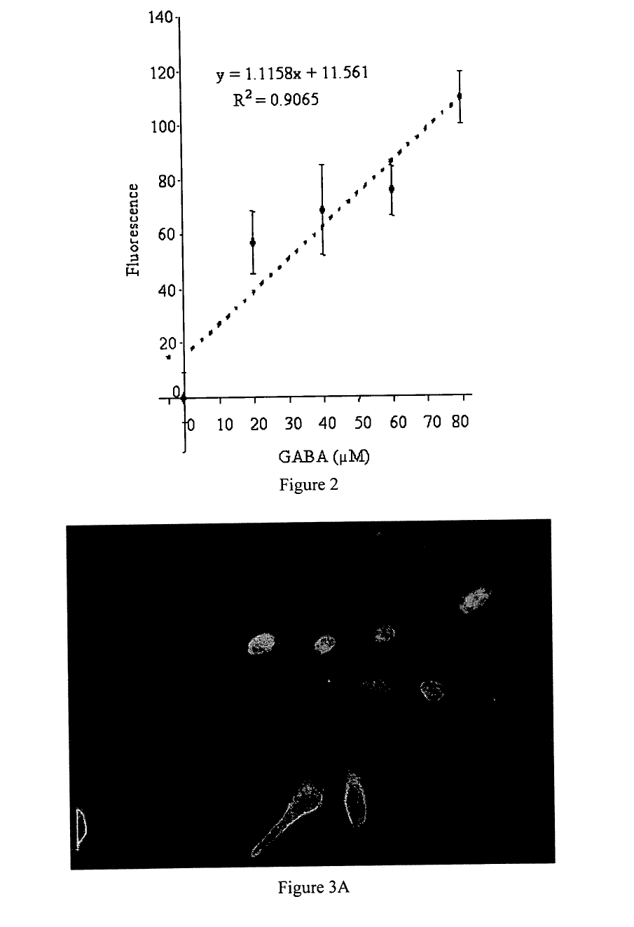

FIG. 2 is a calibration curve showing the amount of fluorescence for GABA using the GABA-T/SSADH silica glass sensor. Because fluorescence may be directly observed, the GABA-T/SSADH silica glass sensor may be used for real-time detection.

Since each silica glass sensor provided linear plots of GABA concentration versus absorbance, the silica glass sensors of the present invention may be used to detect GABA concentrations.

1. CHO Cell Culture on GABA-T/SSADH Silica Glass Sensor

Rat GAD₆₇ (GABA producing enzyme) gene was inserted into vector, pEGFP-N1 (Invitrogen, Carlsbad, Calif.): rGAD₆₇-EGFP. Transient transfection of rGAD₆₇-EGFP was performed with TransFast™ (Promega, Madison, Wis.) based on the manufacturer's directions and methods known in the art. The suspended CHO cells were mixed with DNA and TransFast™ in minimum AMEM EARLE'S medium (Mediatech, Herndon, Va.), which contains 0.51 mM L-glutamic acid, but does not contain GABA or SSA.

The GABA-T/SSADH silica glass sensor was washed with phosphate-buffered saline (PBS, 137 mM NaCl, 2.7 mM KCl, 4.3 mM Na₂HPO₄.7H₂O, 1.4 mM KH₂PO₄) several times after aging and then the surface was coated for 30 minutes with 1 mg/ml Poly-D-lysine (Sigma, St. Louis, Mo.). It is noted that collagen, laminin, or the like may be used in place of Poly-D-lysine. Then suspended Chinese hamster ovary (CHO) cells were cultured on the coated surface in αMEM EARLE'S medium (Mediatech, Herndon, Va.) comprising 30% fetal bovine serum (HyClone, Logan, Utah), 0.5 IU/ml penicillin (Mediatech, Herndon, Va.), and 50 μg/ml streptomycin (Mediatech, Herndon, Va.) with 5% CO₂ at 37° C.

After incubating 24 hours (5% CO₂ at 37° C.), the growth medium was removed with PBS and then CHO cells were treated with Passive Lysis Buffer (Promega, Madison, Wis.) for 15 minutes. Fluorescence of NADPH was measured with Fluoroskan Ascent (Labsystems, Helsinki, Finland).

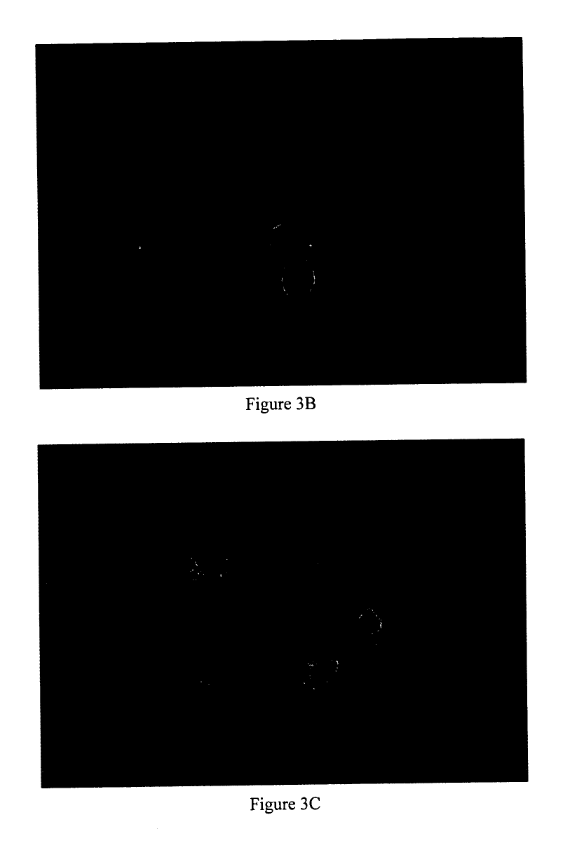

Fluorescence of the EGFP tagged GAD₆₇ indicated that the CHO cells were successfully transfected. FIG. 3A shows EGFP fluorescence from transfected CHO cells on a plastic culture vessel and FIG. 3B shows EGFP fluorescence from vector rGAD₆₇-EGFP transfected CHO cells on the silica glass sensor. FIG. 3C also shows EGFP fluorescence from vector rGAD₆₇-EGFP transfected CHO cells on the silica glass sensor.

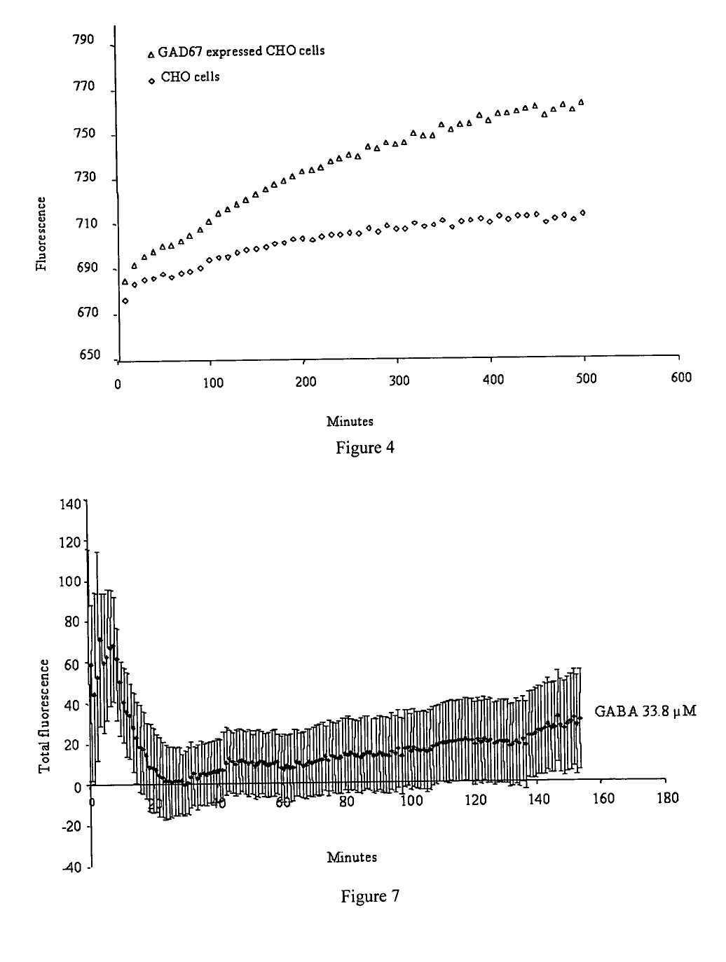

FIG. 4 shows that GABA-T/SSADH silica glass sensor detected the fluorescence from NADPH in real-time, which is generated from GABA degradation reaction by GAD₆₇ in CHO cells. The CHO cells were transfected with vector rGAD-EGFP and GAD₆₇ protein was expressed recombinantly as provided herein. GABA-T/SSADH sensor was prepared and CHO cells were cultured as provided herein.

Cell lysis was performed with Passive Lysis Buffer (Promega, Madison, Wis.) for 15 minutes and added with about 0.1 mM Glutamate, about 0.1 mM α-ketoglutarate, and about 0.14 mM NADP⁺. The reaction was performed at 37° C. and the fluorescence from generated NADPH was measured in-real time.

Since CHO cells have endogenous NADP, NADPH, succinate, SSA, and the like inside the cells, silica glass having SSADH was used as a control to determine whether the fluorescence from the GABA-T/SSADH sensors were the result of GABA produced by GAD67 in the transfected CHO cells. FIG. 5A shows the results. As shown in FIG. 5A, 1:CHO cells on silica glass having SSADH (assayed without α-ketoglutarate and NADP+)=79.19±0.91, 2:CHO cells on silica glass having SSADH (assayed with α-ketoglutarate and NADP+)=99.58±2.52 (2/1=126%), 3:CHO cells cultured on GABA-T/SSADH sensor (assayed without α-ketoglutarate and NADP+)=120±1.12, and 4:CHO cells cultured on GABA-T/SSADH (assayed with α-ketoglutarate and NADP+)=150.13±6.42 ( 4/3=125%).

Then the fluorescence of control CHO cells on silica glass without both GABA-T and SSADH was compared with fluorescence of CHO cells expressing GAD₆₇ on a GABA-T/SSADH sensor using methods known in the art. The control silica glass, which does not encapsulate any enzyme including GABA-T and SSADH, was prepared as according to sol-gel method by Ellerby, et al. and as provided above. The sol was made by mixing 80.7% v/v tetramethylorthosilicate (TMOS >99%) (Aldrich, Milwaukee, Wis.), 18.1% v/v deionized water, and 1.2% v/v HCl (0.04 M), and then sonicating for 15 minutes. The sol was mixed with 0.1M potassium phosphate buffer, pH 6.0 (87.7% 0.2 M KH₂PO₄ and 12.3% 0.2 M K₂HPO₄). The buffered silica sol mixture was poured into a cast. After 30 minutes gelation, the silica glass was washed with phosphate-buffered saline (PBS, 137 mM NaCl, 2.7 mM KCl, 4.3 mM Na₂HPO₄.7H₂O, 1.4 mM KH₂PO₄) and aged at 4° C. for more than about 12 hours. Then the silica glass was washed several times with PBS before use. The amount of TMOS was about 24 μl in total 100 μl of buffered sol.

The results shown in FIG. 5B confirm that the fluorescence from the GABA-T/SSADH sensor is due to GABA produced by GAD₆₇ in CHO cells. CHO cells were very carefully cultured with the same condition during each experiment such that the amount of cells may be assumed to be equal for the silica glass without enzyme and silica glass sensor. As shown in FIG. 5B, 1:CHO cells expressing GAD₆₇ on silica glass/CHO cells on silica glass=99.8%, 2:CHO cells expressing GAD₆₇ on GABA-T/SSADH sensor/CHO cells on GABA-T/SSADH sensor=108.4%. As provided in FIG. 5B, 1 is a control, as it does not encapsulate any enzyme, which shows the comparison of endogenous fluorescence signal between CHO cell and GAD₆₇ expressed CHO cell. In FIG. 5B, 2 shows the comparison of fluorescence signal from NADPH generated from GABA degradation reaction by introduced GAD₆₇ between CHO cell and GAD₆₇ expressed CHO cell.

2. Neural Tissue on GABA-T/SSADH Fluorometric Silica Glass Sensor

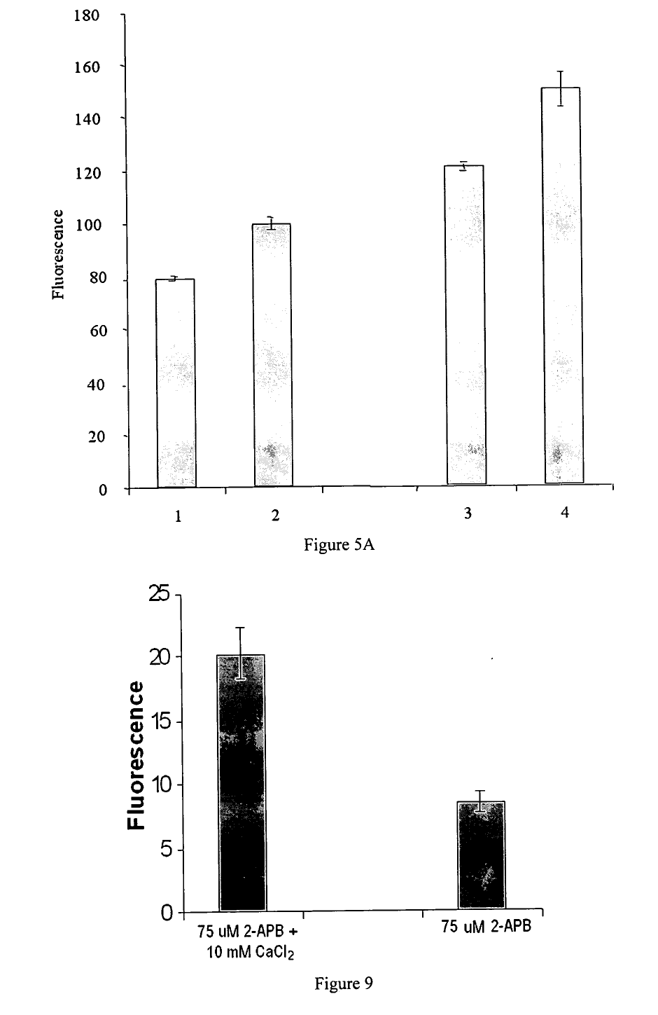

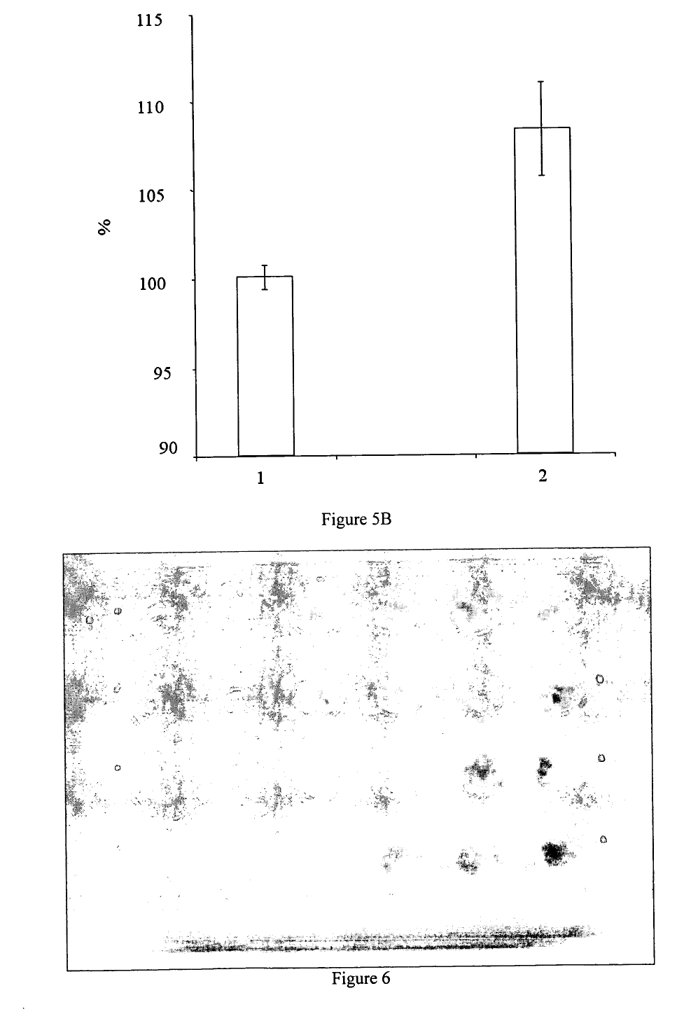

Adult Sprague-Dawley rats about 160 grams each were injected with an overdose of Nembutal. The brains or spinal cords were removed and washed several times with PBS and then embedded with 5% low melting temperature agarose (NuSieve® GAG® agarose, FMC BioProducts, Me.). The embedded brains or spinal cords were cut with a Vibratome (VT10003, LEICA Microsystems, Germany) in 300 μm tissue sections. Each tissue section was placed on a GABA-T/SSADH fluorometric silica glass sensor and then GABA released from the tissue sections were measured with Fluoroskan Ascent (Labsystems, Finland). FIG. 6 shows the GABA-T/SSADH fluorometric silica glass sensor (rat neural tissue sections) and FIG. 7 is a graph of the fluorescence from GABA released from the tissue sections.

As provided herein, the silica glass sensors of the present invention are suitable for assaying GABA, GAD, and SSA. The silica glass sensors comprise encapsulated GABA-T, SSADH, or both and the products of enzymatic conversion may be detected spectrophotometrically or fluorometrically. Specifically, SSA may be assayed spectrophotometrically by reaction with Purpald or fluorometrically based on fluorescence from NADPH. The silica glass sensors of the present invention allow GABA, GAD, and SSA to be assayed in real-time. In preferred embodiments, the fluorescence from NADPH is assayed in real-time. The silica glass sensors also allow GABA, GAD, and SSA from cells and tissues that are cultured on the silica glass sensors to be assayed. Consequently, the silica glass sensors of the present invention may be used to screen agents that affect the GABA pathway in cells cultured on the silica glass sensors. The silica glass sensors of the present invention may also be used to screen and study agents which affect the GABA pathway in various tissues.

Neural Tissue

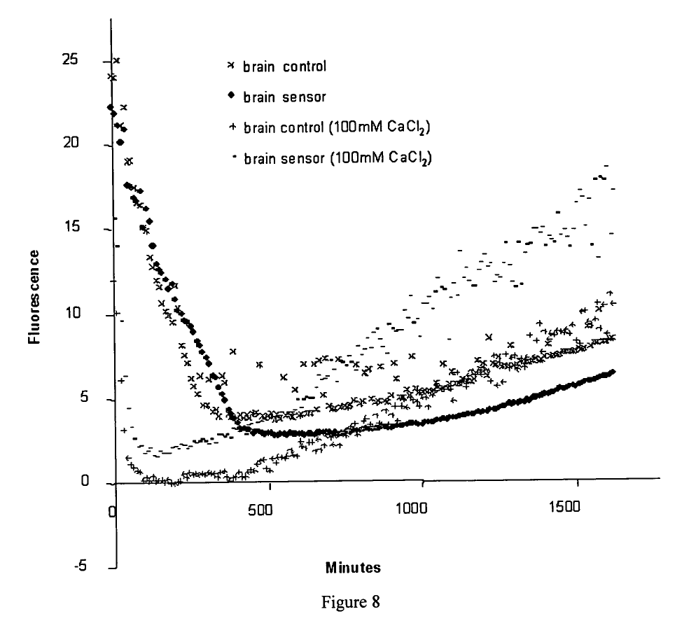

The silica glass sensors of the present invention may be used to assay neural tissue and agents which affect neural tissue. Since glutamate, a major inhibitory neurotransmitter, shows Ca²⁺ dependent release in neural cells, a GABA-T/SSADH sensor was used to detect Ca²⁺-dependent release of GABA from neural cell with neural tissue. Neural tissue, rat brain tissue or rat brain tissue, was cultured on GABA-T/SSADH sensors prepared inside tissue culture vessels. See FIG. 6. Real-time fluorescence detection indicated that at 100 mM Ca²⁺, rat brain tissue released more GABA than controls as provided in FIG. 8. Therefore, GABA release is stimulated by Ca²⁺.

The effect of various agents on GABA release in rat neural tissue were assayed in real-time with a GABA-T/SSADH sensor. The agents tested were 2-APB (a cell-permeable IP3 receptor antagonist), Thapsigargin (a blocker of store-specific Ca²⁺-ATPase), Ryanodine and caffeine (increase internal Ca²⁺ level), and KN-93 (CAM kinase II inhibitor).

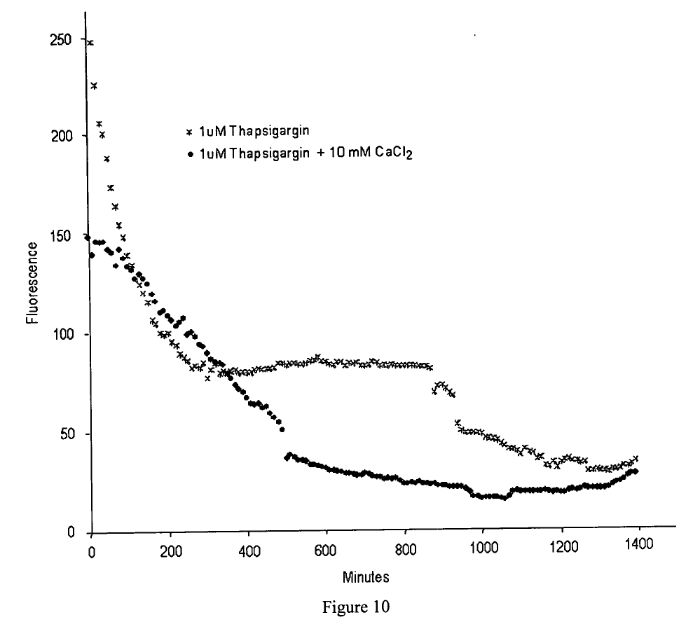

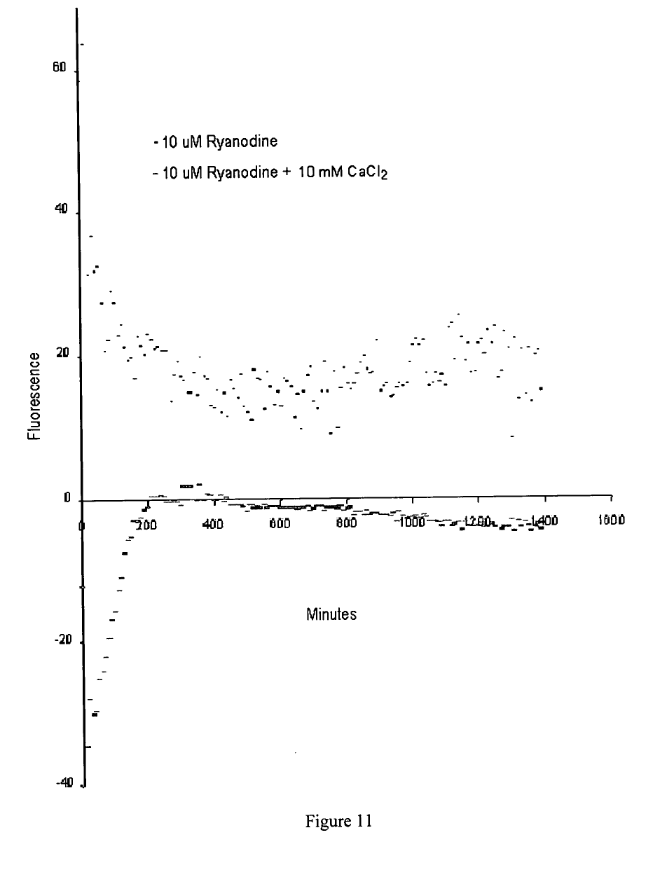

As provided in FIG. 9, 75 μM 2-APB without 10 mM CaCl₂ showed about 41.9% less GABA release than 75 μM 2-APB with 10 mM CaCl₂, thereby indicating that 2-APB inhibits Ca²⁺-dependent GABA release and 10 mM CaCl₂ can modulate the inhibition of 2-APB. FIGS. 10 and 11 show the effect of Thapsigargin and Ryanodine on Ca²⁺-dependent GABA release, respectively. Therefore, the silica glass sensors of the present invention may be used for assaying the effect of various agents on cells and tissues cultured thereon.

As provided herein, the present invention provides encapsulation of more than one agent in a silica glass in one step as a single sol gel layer. Thus, the present invention provides silica glass sensors having two or more agents, such as two or more different enzymes. Silica glass sensors having two or more agents involved in a cellular pathway may be used to screen agents for those that modulate the cellular pathway or detect or diagnose desired activity in the cellular pathway. For example, the mechanisms and agents involved in stem cell differentiation may be analyzed by culturing stem cells on silica glass sensors according to the present invention.

Specifically, cell differentiation is controlled by many factors. Neurotransmitters are related with neural differentiation as well as neurotrophic factors. See Borodinsky, et al. (2004) Nature 429(6991):523-530, which is herein incorporated by reference. Thus, to examine who GABA is involved in cell differentiation, glutamic acid decarboxylase (GAD) may be encapsulated with GABA-T/SSADH (3 enzymes) or with GABA-T (2 enzymes) or without GABA-T/SSADH (1 enzyme) as provided herein. Then stem cells are cultured on the sensors of the present invention for glutamate and GABA (neurotransmitters) which results are correlated to neuron differentiation.

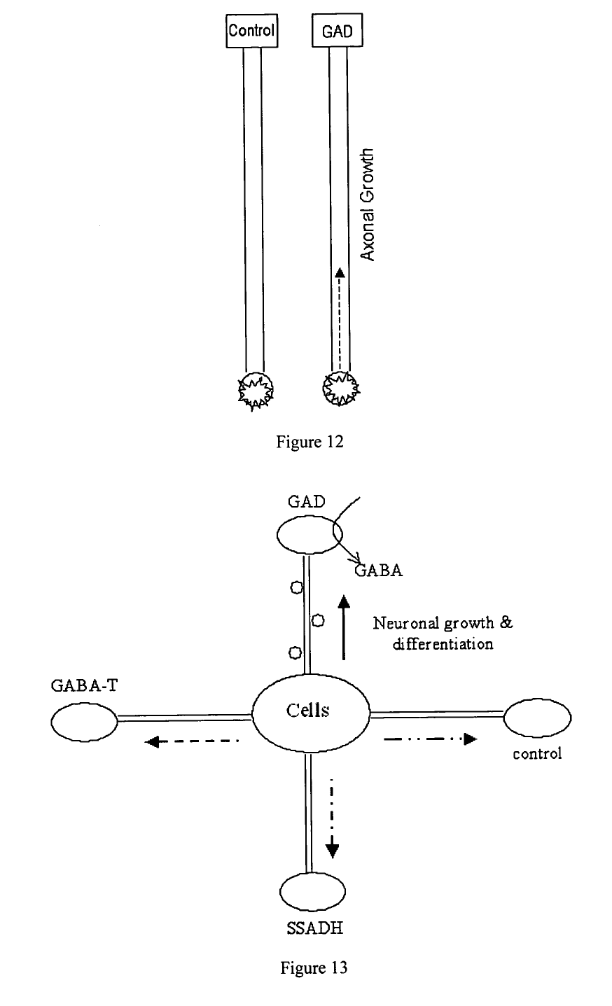

Various neurotransmitters may control neuronal migration, axon formation and neuronal stem cell differentiation. The silica glass sensors of the present invention may be used to study the relationship between various neurotransmitters and metabolites, such as glutamate, GABA, SSA, succinate, and the like, and stem cell differentiation. For example, as provided in FIG. 12, a silica glass sensor, GABA-T or SSADH or GABA-T/SSADH or GAD or GAD/GABA-T or GAD/GABA-T/SSADH, is capsulated in one end of the silica glass sensor and stem cells are cultured at the other end. As the stem cells differentiate, the amount of glutamate, GABA, SSA or succinate may be assayed. FIG. 13 shows an alternative sensor design for conducting comparative studies. In some embodiments, neurotrophic factors, such as BDNF and NGF, may be encapsulated in the silica glass sensors and their effects on cell differentiation and agents which modulate such effects may be studied.

Therefore, as provided herein, the present invention provides methods and devices for assaying (1) mechanisms involved in migration/axon formation, and (2) stem cell development and differentiation including the effects of various agents such as neurotransmitters, pharmaceuticals, metabolites, and the like.

The present invention allows complex enzymatic pathways to be assayed. For example, various enzymes including oxidoreductases, dehydrogenases, kinases, phosphatases, and the like may be encapsulated alone or in combination and fluorescence may be used to detect substrate conversion.

Fluorescence NDPs/NTPs analogs and appropriate proteins can be encapsulated alone or with other biochemically linked enzymes. See e.g. Maruta et al. (2002) J. Biochem. (Tokyo)131(6):905-911, which is herein incorporated by reference. For example, a silica glass sensor of the present invention can be used to detect UDP-transferase related metabolism with fluorescence UDP analog.

Racemase can be encapsulated alone or with other biochemically linked enzymes. The racamase encapsulated sensor detects the content of enatiomer of substrate/product. For example, glutamate racemase catalyzes the interconversion of L-glutamate and D-glutamate. The silica glass sensors of the present invention may be used to measure enatiomeric glutamate. Glutamate racemase, L-glutamate dcarboxylase and GABA-T/SSADH of this invention (GABA-T or GABA-T/SSADH silica sensor) are encapsulated and the contents of L-glutamate/D-glutamate can be measured with the sensor.

Since cells and tissues cultured on the silica glass sensors may be assayed, the present invention allows in vitro and ex vivo analysis of cells and tissues.

Although, for illustrative purposes, the method is described in respect to a particular precursor compound, namely tetramethylorthosilicate (TMOS), and a particular type of active biological material, namely proteins, it is to be understood that the method is not so limited but is also applicable to other silicon alkoxides such as tetraethylorthosilicate (TEOS) and other active silicon compounds. Besides use of other alkoxides of silicon, the invention contemplates the use of other metal alkoxides prepared by adding methanol, ethanol, isopropanol and other similar alcohols to the oxides of various metals and non-metals, including, but not limited to aluminum, titanium, zirconium, niobium, hafnium, chromium, vanadium, tungsten, molybdenum, iron, tin, phosphorus, sodium, calcium, and boron, or combinations thereof.

Additionally, the precursor material or the sol-gel may be tagged by known methods with readily detected substituents, such as optically active groups or constituents which respond to the byproducts of the action of the proteins. Alternatively, other optically active materials may be encapsulated with the protein as indicators of the results of reactions involving the proteins. Other optically active materials include luminescent amino acids, such as tryptophan or other similar materials. Silicon compounds are preferred because silicon chemistry is highly conducive to forming glasses. Among silicon compounds, TMOS is preferred over other materials, such as TEOS, because it reacts faster and does not require alcohol to form a sol.

Further, hydrochloric acid is utilized in the examples but other acids may be utilized to catalyze the reaction between TMOS and water. While HCl is preferred, other suitable acid catalysts include other mineral acids such as sulfuric acid, nitric acid, phosphoric acid, etc. and organic acids such as acetic acid, tartaric acid, phthalic acid, maleic acid, succinic acid and the like and anhydrides of the mineral or organic acids. While acid catalysis is preferred, it is possible to use a base catalyst. However, base catalysts generate rapid gelation, thus making control of the process and the production of monoliths (shaped gels with the smallest dimension greater than a few millimeters) extremely difficult.

Suitable biological materials for encapsulation include, but are not limited to, nucleases, such as RNase A or RNase T1, proteases, such as proteinase K or chymotrypsin, oxidases, such as alcohol oxidase or glucose oxidase, esterases, such as acetylcholine esterase or phosphodiesterase II, isomerases, such as aldolase or glucose isomerase, various proteins including O₂ binders, such as hemoglobin or myoglobin, electron transfer proteins, such as cytochrome c, metal and metal ion binders, such as aequorin, iron and bicarbonate binders, such as transferrin, free radical inhibitors, such as superoxide dismutase and other active biologicals such as ureases. One skilled in the art can readily supplement this list with other biological materials which can be entrapped by the process of the invention; the entrapped material not being a limiting factor. Additionally, the biological materials may be modified or tagged by addition of readily detected substituents such as ions, ligands, optically active groups or other constituents commonly used to tag biological or chemical compounds, suitable luminescent tag include Mn²⁺ or other rare earth metal ions.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

We claim:

1. A porous, transparent sol-gel glass having two or more active biological agents entrapped therein.

2. The sol-gel glass of claim 1, wherein the active biological agents are enzymes.

3. The sol-gel glass of claim 2, wherein the enzymes are involved in a given cellular pathway.

4. The sol-gel glass of claim 1, wherein the active biological agents are GABA-transaminase (GABA-T) and SSA-dehydrogenase (SSADH).

5. A method for assaying a test sample for a substance which reacts with or whose reaction is catalyzed by an active biological material which comprises
contacting a porous, transparent sol-gel glass having the biological material entrapped therein into contact with the test sample; and
observing any change in the optical characteristics of the porous, transparent sol-gel glass.

6. The method of claim 5, wherein the change in optical characteristics is observed using spectroscopic techniques selected from the group consisting of ultraviolet, infrared, visible light, fluorescence, luminescence, absorption, emission and reflection techniques.

7. The method of claim 5, wherein the active biological material is GABA-transaminase (GABA-T), SSA-dehydrogenase (SSADH), or both.

8. The method of claim 5, wherein the test sample is a cell or a tissue.

9. The method of claim 8, wherein the test sample is a neuron or a stem cell.

10. The method of claim 8, wherein the cell or tissue is cultivated on the porous, transparent sol-gel glass having the biological material entrapped therein.

11. The method of claim 5, wherein the substance is γ-aminobutyric acid (GABA), glutamic acid dehydrogenase (GAD), succinic semialdehyde (SSA), or a combination thereof.

12. An assay for a substrate involved in the γ-aminobutyric acid (GABA) pathway in a sample which comprises contacting the sample with an enzyme for the substrate and observing conversion of the substrate.

13. The assay of claim 12, wherein the substrate is γ-aminobutyric acid (GABA), or succinic semialdehyde (SSA).

14. The assay of claim 12, wherein the enzyme is GABA-transaminase (GABA-T) or SSA-dehydrogenase (SSADH).

15. The assay of claim 12, wherein conversion of the substrate is observed by assaying the amount NADPH produced after the enzyme is contacted with the substrate.

16. The assay of claim 12, wherein the substrate is γ-aminobutyric acid (GABA) and conversion is observed by assaying the amount of succinic semialdehyde (SSA) produced after the enzyme is contacted with the substrate.

17. The assay of claim 16, wherein the amount of succinic semialdehyde (SSA) is assayed by reacting with 4-amino-3-hydrazino-5-mercapto-1, 2, 3-triazole.


SEQUENCE LISTING (4 sequences)

SEQ ID NO: 1 - DNA, 29 bp, Artificial
  oligonucleotides for gabT (GABA-T) PCR reaction
cccggatccatgaacagcaataaagagtt

SEQ ID NO: 2 - DNA, 27 bp, Artificial
  oligonucleotides for gabT (GABA-T) PCR reaction
cccaagcttctactgcttcgcctcatc

SEQ ID NO: 3 - DNA, 29 bp, Artificial
  oligonucleotides for gabD (SSADH) PCR reaction
cccggatccatgaaacttaacgacagtaa

SEQ ID NO: 4 - DNA, 29 bp, Artificial
  oligonucleotides for gabD (SSADH) PCR reaction
cccaagcttttaaagaccgatgcacatat